(19) United States
(12) Patent Application Publication
(10) Pub. No.: US 2008/0107726 A1
(43) Pub. Date: May 8, 2008

(54) COMPOSITIONS COMPRISING BETA-ADRENERGIC RECEPTOR ANTAGONISTS AND DIURETICS

(76) Inventors: Pramod Kharwade, Chhindwara (IN); Indu Bhushan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/930,467

(22) Filed: Oct. 31, 2007

Related U.S. Application Data
(60) Provisional application No. 60/890,212, filed on Feb. 16, 2007.

(30) Foreign Application Priority Data
Nov. 1, 2006 (IN) 2034/CHE/2006

(51) Int. Cl. A61K 9/20; A61K 9/50; A61K 9/48; A61K 9/26; A61K 47/30; A61K 31/135; A61K 31/535; A61P 43/00

(52) U.S. Cl. 424/451; 424/497; 514/772.3; 514/651; 514/233.5; 424/464; 424/470

(57) ABSTRACT

Pharmaceutical compositions comprise beta-adrenergic receptor blocking agents for controlled release and diuretics for immediate release. In an embodiment, a composition comprising metoprolol succinate is coated onto inert particles, and the particles are further coated with a polymer to provide controlled release of metoprolol. The coated particles are coated, granulated, or mixed with a composition comprising hydrochlorothiazide, then are compressed into tablets, or the coated particles are compressed into tablets and the tablets are coated with a composition comprising hydrochlorothiazide.

### INTRODUCTION TO THE INVENTION

The present invention relates to pharmaceutical compositions comprising beta-adrenergic receptor blocking agents and diuretics. More particularly, the present invention relates to pharmaceutical compositions comprising beta-adrenergic receptor blocking agents for controlled release and diuretics for immediate release.

Metoprolol, a β₁-cardioselective adrenergic receptor antagonist drug, is a highly lipophilic compound having a log P of 2.48. The metoprolol succinate salt has a chemical name (±)-1-(isopropylamino)-3-[p-(2-methoxyethyl)phenoxy]-2-propanol succinate (2:1) (salt). It is freely soluble in water and useful in the treatment of hypertension, angina pectoris and heart failure. Metoprolol succinate is a white crystalline powder with a molecular weight of 652.8. It is freely soluble in water, soluble in methanol, sparingly soluble in ethanol, slightly soluble in dichloromethane and 2-propanol, and practically insoluble in ethyl acetate, acetone, diethylether and heptane. The structural formula for metoprolol succinate is Formula I.

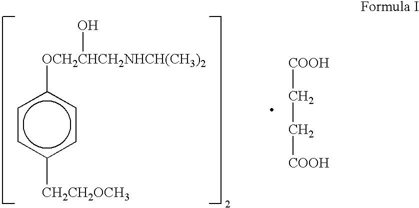

Hydrochlorothiazide is a thiazide diuretic having a chemical name 6-chloro-3,4-dihydro-2H-1,2,4-benzothiadiazine-7-sulfonamide 1,1-dioxide. It is a white, or practically white, crystalline powder with a molecular weight of 297.7, and is slightly soluble in water but freely soluble in sodium hydroxide solution. Structurally, hydrochlorothiazide is represented as Formula II.

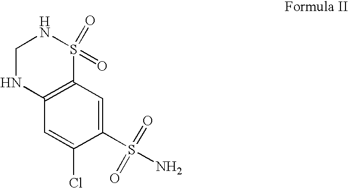

Metoprolol salts and hydrochlorothiazide have been used individually and in combination for the treatment of hypertension. The antihypertensive effects of these agents are additive. Products sold as DUTOPROL™ (metoprolol succinate extended release/hydrochlorothiazide) combine metoprol succinate and hydrochlorothiazide, and are manufactured by AstraZeneca. DUTOPROL™ products are available for oral administration in three tablet strengths of metoprolol succinate extended release and hydrochlorothiazide (HCTZ) immediate release (equivalent to 25 mg metoprolol tartrate/12.5 mg HCTZ, equivalent to 50 mg metoprolol tartrate/12.5 mg HCTZ, and equivalent to 100 mg metoprolol tartrate/12.5 mg HCTZ). The latter product is also sold in Brazil as SELOPRESS ZOK®.

U.S. Pat. Nos. 4,927,640 and 4,957,745, and U.S. Patent Application Publication Nos. 2005/0008701 and 2003/0185887, describe controlled release preparations of metoprolol salts.

U.S. Pat. No. 4,957,745 and International Application Publication Nos. WO 2004/069234 and WO 2003/086353 describe extended release metoprolol compositions.

U.S. Patent Application Publication No. 2005/032879 discloses formulations comprising a beta-blocker and an ACE inhibitor.

U.S. Pat. No. 6,252,113 describes the manufacturing process for metoprolol.

The pharmaceutical compositions of the present invention were surprisingly found to exhibit desired in vitro release and in vivo absorption profiles. Thus, the present invention provides much-needed pharmaceutical compositions comprising beta-adrenergic receptor blocking agents for controlled release and diuretics for immediate release, as an economical alternative to the currently marketed products.

### SUMMARY OF THE INVENTION

An aspect of the present invention provides for pharmaceutical compositions comprising beta-adrenergic receptor blocking agents for controlled release and diuretics for immediate release.

Another aspect of the present invention provides pharmaceutical compositions comprising metoprolol or its pharmaceutically acceptable salts, solvates, polymorphs, enantiomers, single isomer, or mixtures thereof for controlled release, and hydrochlorothiazide or its pharmaceutically acceptable salts, solvates, polymorphs, enantiomers, single isomer, or mixtures thereof for immediate release.

In one embodiment of the invention, a controlled release component of the composition comprises seed cores and metoprolol or its pharmaceutically acceptable salt, optionally with one or more hydrophilic or hydrophobic polymers or mixtures thereof, deposited or layered or applied onto the seed cores.

In another embodiment of the invention, an immediate release component of the composition comprises hydrochlorothiazide and at least one pharmaceutical excipient, wherein said immediate release component is:

a. deposited or layered or applied onto the controlled release component;

b. granulated with the controlled release component;

c. mixed with the controlled release component; and/or

d. applied as a coating onto a pharmaceutical composition comprising the controlled release component.

In an embodiment, the invention provides a pharmaceutical formulation comprising:
a) a plurality of inert particles having a coating comprising a β-adrenergic receptor antagonist drug and a hydrophilic polymer, hydrophobic polymer, or combination of hydrophilic and hydrophobic polymers, providing controlled release of drug; and
b) a diuretic.

In another embodiment, the invention provides a pharmaceutical formulation comprising:
a) a plurality of substantially water-insoluble inert particles having a coating comprising metoprolol or a salt thereof and a hydrophilic polymer, hydrophobic polymer, or combination of hydrophilic and hydrophobic polymers, providing controlled release of metoprolol; and
b) a diuretic.

In a further embodiment, the invention provides a pharmaceutical formulation comprising:
a) a plurality of substantially water-insoluble inert particles having a coating comprising metoprolol succinate and a hydrophilic polymer, hydrophobic polymer, or combination of hydrophilic and hydrophobic polymers, providing controlled release of metoprolol; and
b) hydrochlorothiazide in an immediate release form.

### DETAILED DESCRIPTION

The present invention relates to pharmaceutical compositions comprising beta-adrenergic receptor blocking agents for controlled release and diuretics for immediate release.

“Controlled release” is used herein to describe dissolution of a drug that occurs in a manner differing from the immediate release obtained from formulations that do not contain polymers that affect drug dissolution. Controlled release can be obtained by delaying dissolution of the drug, such as by incorporating polymers into a formulation that resist dissolution in certain pH regimes, incorporating polymers that modify the rate at which drug dissolves from a formulation, or any combination thereof.

In accordance with the invention, the term “seed cores” may also termed as inert cores, inert particles, inert spheres, starter cores, etc.

Typical non-limiting examples of beta-adrenergic receptor blocking agents in the context of the present invention include alprenolol, metoprolol, atenolol, propanolol, acebutolol, bisoprolol, nodolol, sotalol, timolol, esmolol, labetalol, pindolol, and the like.

Typical non-limiting examples of diuretics in the context of the present invention include hydrochlorothiazide, bendroflumethiazide, caffeine, theophylline, amiloride, spiranolactone, triamterene, bumetanide, acetazolamide, dorzolamide, and the like.

The mention of a drug compound is intended to include salts and other compounds thereof that can be used in pharmaceutical formulations to provide the drug in solutions.

In an aspect of present invention, a controlled release component of the composition comprises seed cores and metoprolol or its pharmaceutically acceptable salt, optionally with one or more hydrophilic or hydrophobic polymer or mixtures thereof, deposited or layered or applied onto the seed cores.

Particular embodiments of this invention relate to pharmaceutical compositions having:

a. seed cores;

b. optionally, a seal coating on the seed cores;

c. metoprolol or its pharmaceutically acceptable salt, optionally with one or more hydrophilic or hydrophobic polymers including mixtures thereof, deposited or layered or applied onto the seed cores;

d. optionally, an outer coating of a polymer blend utilizing groups of polymers having opposing wettability characteristics that releases metoprolol or its pharmaceutically acceptable salt substance in a controlled manner over a period of time; and

e. hydrochlorothiazide or its pharmaceutically acceptable salt, for immediate release.

In embodiments of the invention, an immediate release component of the compositions comprises hydrochlorothiazide and at least one pharmaceutical excipient, wherein said immediate release component is:

a. deposited or layered or applied onto a controlled release component;

b. granulated with a controlled release component;

c. mixed with a controlled release component; and/or

d. applied as a coating on a pharmaceutical composition comprising a controlled release component.

The compositions comprise a large number of small seed cores having sizes ranging from about 50 to about 5000 μm, or from about 100 to about 500 μm, or from about 150 to about 300 μm. The cores are pharmacologically inert in nature and pharmaceutically compatible. Non-limiting examples of various substances that can be used as seed cores include insoluble inert materials such as glass particles/beads or silicon dioxide, calcium phosphate dihydrate, dicalcium phosphate, calcium sulfate dihydrate, microcrystalline cellulose, cellulose derivatives, calcium carbonate, dibasic calcium phosphate anhydrous, dibasic calcium phosphate monohydrate, tribasic calcium phosphate, magnesium carbonate, and magnesium oxide, soluble cores such as sugar spheres having sugars like dextrose, lactose, anhydrous lactose, spray-dried lactose, lactose monohydrate, mannitol, starches, sorbitol, and sucrose, insoluble inert plastic materials such as spherical or nearly spherical core beads of polyvinylchloride or polystyrene, and any other pharmaceutically acceptable insoluble synthetic polymeric materials, and the like and mixtures thereof.

In one embodiment, dibasic calcium phosphate anhydrous has been found to be particularly useful as an inert water-insoluble inorganic seed core for the present invention. Dibasic calcium phosphate is a widely used inorganic pharmaceutical excipient, which is available in two forms, hydrous and anhydrous. Depending on the moisture sensitivity of the drug, any of the forms can be chosen.

The present invention in one embodiment provides metoprolol on inert water-insoluble seed cores, coated with a hydrophilic or hydrophobic coating polymer, or a mixture thereof, to obtain a defined coating built up. Such hydrophilic polymers of various grades are exemplified, but are not limited to, celluloses such as hydroxypropyl cellulose, carboxymethyl cellulose sodium, hydroxyethyl cellulose, hydroxypropyl methylcellulose (HPMC), homopolymers or copolymers of N-vinylpyrrolidone, vinyl and acrylic polymers, polyacrylic acid and the like, hydrophobic polymers such as celluloses like ethyl cellulose, cellulose acetate, cellulose propionate (lower, medium or higher molecular weight), cellulose acetate propionate, cellulose acetate butyrate, and cellulose acetate phthalate, polyalkyl methacrylates, polyalkyl acrylates, polyvinyl acetate (PVA), chitosan, stearic acid, gum arabic, crosslinked vinylpyrrolidone polymers, hydrogenated castor oil, and the like. Other classes of release controlling substances or their mixtures in various ratios as required are also within the purview of this invention without limitation.

In one of the embodiments, polymers such as hydroxypropyl methylcellulose have been found particularly useful for the coating composition in combination with hydrophobic polymers such as ethyl cellulose to modulate the release of the metoprolol in a predictable controlled manner for a prolonged or sustained period of time.

In an embodiment of the present invention, a weight ratio of hydrophilic to hydrophobic polymer for the coating composition ranges from about 1:01 to about 1:9, or from about 1:1 to about 1:3, respectively.

In one of the embodiments a method of preparing a pharmaceutical composition includes, but is not limited to, one or more of physical mixing, blending, dry granulation, wet granulation, and direct compression.

In an embodiment of the invention, compositions are prepared as follows:

a. optionally coating a hydrophilic or hydrophobic polymer onto seed cores;

b. layering metoprolol, optionally with one or more hydrophilic or hydrophobic polymer or mixtures thereof, onto inorganic seed cores or seal coated seed cores;

c. optionally coating the metoprolol layer with one or more hydrophilic or hydrophobic polymers or a mixture thereof; and

d. coating the controlled release pellets comprising metoprolol of step b) or c) with a coating composition comprising hydrochlorothiazide, granulating the pellets and further compressing into tablets or filling into capsules; or

e. granulating the controlled release pellets comprising metoprolol of step b) or c), lubricating the granulate with a mixture comprising hydrochlorothiazide, and compressing into tablets or filling into capsules; or

f. mixing the controlled release pellets comprising metoprolol of step b) or c) and hydrochlorothiazide, optionally with pharmaceutical excipients, and granulating, and further compressing into tablets or filling into capsules; or

g. granulating the controlled release pellets comprising metoprolol of step b) or c) and compressing into tablets, and coating the tablets with a film coating composition comprising hydrochlorothiazide.

In embodiments, the controlled release metoprolol component of the composition can be in the form of pellets. This controlled release metoprolol component and an immediate release hydrochlorothiazide component can be compressed into tablets or filled into hard gelatin capsules, sachets and the like so to obtain the desired in vitro release and in vivo absorption profiles.

The equipment suitable for processing the pharmaceutical composition of the present invention include mechanical sifters, fluid bed granulators (FBG), fluid bed coaters (FBC), blenders, roller compacters, compression machines, rotating bowls or coating pans, etc.

In the context of the present invention, during the preparation of the pharmaceutical compositions into finished dosage forms, one or more pharmaceutically acceptable excipients may optionally be used, including but not limited to: diluents such as microcrystalline cellulose (MCC), silicified MCC (e.g. Prosolv™ HD 90), microfine cellulose, lactose, starch, pregelatinized starch, polyethylene Glycol, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and the like; binders such as acacia, guar gum, alginic acid, dextrin, maltodextrin, methylcellulose, ethyl cellulose, hydroxyethyl cellulose, hydroxypropyl cellulose (e.g. KLUCEL®), hydroxypropyl methylcellulose (e.g. METHOCEL®), carboxymethyl cellulose sodium, povidone (various grades of KOLLIDON®, PLASDONE®), starch and the like; disintegrants such as carboxymethyl cellulose sodium (e.g. Ac-Di-Sol®, PRIMELLOSE®), crospovidone (e.g. KOLLIDON®, POLYPLASDONE®), povidone K-30, polacrilin potassium, starch, pregelatinized starch, sodium starch glycolate (e.g. EXPLOTAB®) and the like; plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol, and the like. Solvents that may be used in processing, including steps such as granulation or layering or coating include water, methanol, ethanol, isopropyl alcohol, acetone, methylene chloride, and the like, and mixtures thereof.

Pharmaceutical compositions of the present invention may further include any one or more of pharmaceutically acceptable glidants, lubricants, opacifiers, colorants and other commonly used excipients.

The pharmaceutical compositions as disclosed in the context of the present invention are useful in the treatment of hypertension.

The tablets/capsules or any other dosage forms prepared as above can be subjected to an in vitro dissolution evaluation according to Test 711 “Dissolution” in United States Pharmacopoeia 29, United States Pharmacopeial Convention, Inc, Rockville, Md., 2005 to determine the rate at which the active substances are released from the dosage forms, and content of active substance can be determined in solutions using techniques such as high performance liquid chromatography. The pharmaceutical dosage forms of the present invention are intended for oral administration to a patient in need thereof.

In an embodiment the invention includes the use of packaging materials such as containers and lids of high-density polyethylene (HDPE), low-density polyethylene (LDPE) and or polypropylene and/or glass, and blisters or strips composed of aluminium of high-density polypropylene, polyvinyl chloride, polyvinylidene dichloride, etc.

The following examples are included only to further illustrate certain specific aspects and embodiments of the invention, and are not to be construed as limiting the scope of the invention.

### EXAMPLE 1

### Preparation of Metoprolol Succinate Controlled Release Pellets

| Ingredient | Grams |
|---|---|
| Dibasic calcium phosphate anhydrous (A-Tab)* | 100 |

SEAL COATING

| | |
|---|---|
| Ethyl cellulose, 10 cps | 12 |
| Acetyltributyl citrate | 3 |
| Isopropyl alcohol ‡ | 180 |
| Dichloromethane ‡ | 95 |

METOPROLOL LAYER

| | |
|---|---|
| Seal coated pellets | 76 |
| Metoprolol succinate** | 380 |
| Hypromellose, 5 cps | 44 |
| Water ‡ | 788 |

CONTROLLED RELEASE COATING

| | |
|---|---|
| Ethyl cellulose, 10 cps | 240 |
| Hypromellose, 5 cps | 52 |
| Acetyltributyl citrate | 58 |
| Isopropyl alcohol ‡ | 4433 |
| Dichloromethane ‡ | 2217 |

*A-Tab is a powder form manufactured by Rhodia.
**Amount expressed as the metoprolol tartrate equivalent.
‡ Evaporates during processing.

Manufacturing Process:

1. Ethyl cellulose and acetyltributyl citrate were dispersed in a mixture of isopropyl alcohol and methylene chloride.

2. The dispersion of step 1 was coated onto dibasic calcium phosphate using a fluidized bed coater (FBC) to produce a 15% weight gain.

3. Metoprolol succinate and hypromellose were dissolved in water to form a solution.

4. The drug solution of step 3 was coated onto seal coated cores of step 2 using a FBC to produce a 558% weight gain.

5. Ethyl cellulose, hypromellose and acetyltributyl citrate were dispersed in a mixture of isopropyl alcohol and methylene chloride.

6. The coating solution of step 5 was coated onto drug loaded pellets of step 4 using a FBC to produce a 70% weight gain.

### EXAMPLES 2-5

### Compositions for Metoprolol 100 mg Controlled Release and Hydrochlorothiazide 12.5 mg Tablets.

| | Example 2 | Example 3 | Example 4 | Example 5 |
|---|---|---|---|---|
| Ingredient | Grams | | | |
| Metoprolol succinate ER | — | — | — | 106.3 |
| pellets | | | | |

HYDROCHLOROTHIAZIDE LAYERING

| | | | | |
|---|---|---|---|---|
| Metoprolol succinate ER | — | — | — | 106.3 |
| pellets | | | | |
| Hydrochlorothiazide | — | — | — | 7.5^{@} |
| Polyethylene glycol 6000 | — | — | — | 7.5^{@} |
| Water ‡ | — | — | — | 285^{@} |

GRANULATION

| | | | | |
|---|---|---|---|---|
| Metoprolol succinate ER | 106.3 | 106.3 | 106.3 | 118.8 |
| pellets | | | | |
| Prosolv HD 90* | 150 | 150 | 104.1 | 104.1 |
| Hydroxypropyl cellulose | 12 | 12.6^{@} | 10.1 | 10.1 |
| Hydrochlorothiazide | — | 6.56^{@} | — | — |
| Water ‡ | | 231.5^{@} | 190 | 190 |

BLENDING AND LUBRICATION

| | | | | |
|---|---|---|---|---|
| Hydroxypropyl cellulose | 4.7 | 4.7 | 7.5 | 7.5 |
| Croscarmellose sodium | 5.9 | 5.9 | 5.9 | 5.9 |
| Sodium stearyl fumarate | 1.2 | 1.2 | 1.2 | 1.2 |
| Hydrochlorothiazide | 6.3 | — | — | — |

FILM COATING

| | | | | |
|---|---|---|---|---|
| Hypromellose, 5 cps | — | — | 5^{@} | — |
| Polyethylene glycol 6000 | — | — | 7.5^{@} | — |
| Talc | — | — | 0.6^{@} | — |
| Titanium dioxide | — | — | 5^{@} | — |
| Hydrochlorothiazide | — | — | 7.5^{@} | — |
| Isopropyl alcohol ‡ | — | — | 242@ | — |
| Dichloromethane ‡ | — | — | 242@ | — |

*Silicified microcrystalline cellulose (or co-processed MCC with silicon dioxide), JRS Pharma GmbH Co. KG, Rosenberg, Germany.
^{@}Contains 20% excess to account for processing losses.
‡ Evaporates during processing.

Manufacturing Processes:

### EXAMPLE 2

1. Metoprolol succinate pellets of Example 1 were mixed with Prosolv.

2. Blend of step 1 was granulated with aqueous solution of hydroxypropyl cellulose solution using a fluidized bed processor (FBP).

3. Granules of step 2 were blended with hydroxypropyl cellulose, croscarmellose sodium, sodium stearyl fumarate and hydrochlorothiazide.

4. Blend of step 3 was compressed into tablets using a 11 mm round punch set.

### EXAMPLE 3

1. Metoprolol succinate pellets of Example 1 were mixed with Prosolv.

2. Blend of step 1 was granulated with a hydroxypropyl cellulose and hydrochlorothiazide suspension in water using a fluidized bed processor (FBP).

3. Blending (except hydrochlorothiazide) and compression processes were similar to that described in Example 2.

### EXAMPLE 4

1. Mixing, granulation, blending (except hydrochlorothiazide) and compression processes were similar to those described in Example 2.

2. Tablets were coated with a dispersion of hypromellose, polyethylene glycol, talc, titanium dioxide and hydrochlorothiazide in the mixture of isopropyl alcohol and dichloromethane, to produce a 9% weight build-up.

The in vitro dissolution profile of the Example 4 product was compared with that for the commercial product SELOPRESS ZOK® and results are given in Table 1 for metoprolol succinate and hydrochlorothiazide release.

Dissolution Conditions:

Media: pH 6.8 phosphate buffer.

Apparatus: USP apparatus II (Paddle) from Test 711 “Dissolution” in United States Pharmacopeia 29, United States Pharmacopeial Convention, Inc., Rockville, Md., 2005.

Stirring speed: 50 rpm.

Volume: 900 ml.

TABLE 1

| Time | SELOPRESS ZOK ® | Example 4 |
|---|---|---|
| | Cumulative % Hydrochlorothiazide | |
| (Minutes) | Dissolved | |
| 15 | 67 | 53 |
| 30 | 74 | 81 |
| 45 | 76 | 93 |
| 60 | 77 | 114 |
| (Hours) | Cumulative % Metoprolol Dissolved | |
| 1 | 6 | 14 |
| 4 | 20 | 39 |
| 8 | 43 | 55 |
| 12 | 65 | 65 |
| 20 | 88 | 83 |

### EXAMPLE 5

1. Metoprolol succinate pellets of Example 1 were coated with a dispersion of hydrochlorothiazide and polyethylene glycol in water.

2. Pellets of step 1 were mixed, granulated, blended and compressed by processes similar to those described in Example 2.

### EXAMPLE 6

### Composition for Metoprolol 100 mg Controlled Release and Hydrochlorothiazide 12.5 mg Tablets.

| Ingredient | Grams |
|---|---|
| Dibasic calcium phosphate anhydrous (A-Tab) | 100 |

SEAL COATING

| | |
|---|---|
| Ethyl cellulose, 10 cps | 12 |
| Acetyltributyl citrate | 3 |
| Isopropyl alcohol ‡ | 180 |
| Dichloromethane ‡ | 95 |

METOPROLOL LAYER

| | |
|---|---|
| Seal coated pellets | 76 |
| Metoprolol succinate* | 380 |
| Hypromellose, 5 cps | 44 |
| Water ‡ | 788 |

EXTENDED RELEASE COATING

| | |
|---|---|
| Ethyl cellulose, 10 cps | 291.4 |
| Hypromellose, 5 cps | 63.2 |
| Acetyltributyl citrate | 70.4 |
| Isopropyl alcohol ‡ | 4680 |
| Dichloromethane ‡ | 2340 |
| Water ‡ | 1050 |

GRANULATION

| | |
|---|---|
| Metoprolol succinate ER pellets | 115.6 |
| Prosolv HD 90 | 140.6 |
| Hydroxypropyl cellulose | 12 |
| Water ‡ | 190 |

BLENDING AND LUBRICATION

| | |
|---|---|
| Hydrochlorothiazide | 6.3 |
| Hydroxypropyl cellulose | 4.7 |
| Croscarmellose sodium | 5.9 |
| Sodium stearyl fumarate | 1.2 |

*Amount expressed as the metoprolol tartarate equivalent.
‡ Evaporates during processing.

Manufacturing process was the same as for Example 3.

In vitro dissolution profiles of products from Examples 2, 3, 5, and 6 were compared with that for the commercial product SELOPRESS ZOK® and drug release results are given in Table 2 for metoprolol succinate and in Table 3 for hydrochlorothiazide.

Dissolution Conditions:

Media: pH 6.8 phosphate buffer.

Apparatus: USP apparatus II (Paddle) from Test 711 “Dissolution” in United States Pharmacopeia 29, United States Pharmacopeial Convention, Inc., Rockville, Md., 2005.

Stirring speed: 100 rpm.

Volume: 900 ml.

TABLE 2

| | Cumulative % Metoprolol Dissolved | | | | |
|---|---|---|---|---|---|
| Time | SELOPRESS | Example | Example | Example | Example |
| (hours) | ZOK ® | 2 | 3 | 5 | 6 |
| 1 | 5 | 20 | 25 | 27 | 9 |
| 4 | 25 | 42 | 49 | 51 | 25 |
| 8 | 51 | 62 | 70 | 67 | 37 |
| 12 | 76 | 86 | 88 | 83 | 52 |
| 20 | 98 | 92 | 102 | 95 | 82 |

TABLE 3

| Time | Cumulative % Hydrochlorothiazide Dissolved | | | | |
|---|---|---|---|---|---|
| (min- | SELOPRESS | Example | Example | Example | Example |
| utes) | ZOK ® | 2 | 3 | 5 | 6 |
| 15 | 93 | 96 | 104 | 83 | 82 |
| 30 | 95 | 95 | 104 | 83 | 82 |
| 45 | 96 | 94 | 104 | 83 | 83 |
| 60 | 96 | 94 | 104 | 83 | 83 |

We claim:

1. A pharmaceutical formulation comprising:
a) a plurality of inert particles having a coating comprising a β-adrenergic receptor antagonist drug and a hydrophilic polymer, hydrophobic polymer, or combination of hydrophilic and hydrophobic polymers, providing controlled release of drug; and
b) a diuretic.

2. The pharmaceutical formulation of claim 1, wherein inert particles are substantially insoluble in water.

3. The pharmaceutical formulation of claim 1, wherein inert particles comprise a calcium phosphate.

4. The pharmaceutical formulation of claim 1, wherein particles of a) are coated with a composition comprising b).

5. The pharmaceutical formulation of claim 1, wherein particles of a) are granulated with a composition comprising b).

6. The pharmaceutical formulation of claim 1, wherein particles of a) are mixed with b) and optionally one or more pharmaceutical excipients, then granulated.

7. The pharmaceutical formulation of claim 1, wherein particles of a), optionally with one or more pharmaceutical excipients, are compressed into tablets and the tablets are coated with a composition comprising b).

8. The pharmaceutical formulation of claim 1, wherein a β-adrenergic receptor antagonist drug comprises metoprolol or a salt thereof.

9. The pharmaceutical formulation of claim 1, wherein a β-adrenergic receptor antagonist drug comprises metoprolol succinate.

10. The pharmaceutical formulation of claim 1, wherein a diuretic comprises hydrochlorothiazide.

11. The pharmaceutical formulation of claim 1, wherein β-adrenergic receptor antagonist drug comprises metoprolol succinate and a diuretic comprises hydrochlorothiazide.

12. The pharmaceutical formulation of claim 1, being compressed into tablets.

13. The pharmaceutical formulation of claim 1, wherein particles are filled into capsules.

14. A pharmaceutical formulation comprising:
a) a plurality of substantially water-insoluble inert particles having a coating comprising metoprolol or a salt thereof and a hydrophilic polymer, hydrophobic polymer, or combination of hydrophilic and hydrophobic polymers, providing controlled release of metoprolol; and
b) a diuretic.

15. The pharmaceutical formulation of claim 14, wherein inert particles comprise a calcium phosphate.

16. The pharmaceutical formulation of claim 14, wherein particles of a) are coated with a composition comprising b).

17. The pharmaceutical formulation of claim 14, wherein particles of a) are granulated with a composition comprising b).

18. The pharmaceutical formulation of claim 14, wherein particles of a) are mixed with b) and optionally one or more pharmaceutical excipients, then granulated.

19. The pharmaceutical formulation of claim 14, wherein particles of a), optionally with one or more pharmaceutical excipients, are compressed into tablets and the tablets are coated with a composition comprising b).

20. The pharmaceutical formulation of claim 14, wherein metoprolol or a salt thereof comprises metoprolol succinate.

21. The pharmaceutical formulation of claim 14, wherein a diuretic comprises hydrochlorothiazide.

22. The pharmaceutical formulation of claim 14, wherein metoprolol or a salt thereof comprises metoprolol succinate and a diuretic comprises hydrochlorothiazide.

23. A pharmaceutical formulation comprising:
a) a plurality of substantially water-insoluble inert particles having a coating comprising metoprolol succinate and a hydrophilic polymer, hydrophobic polymer, or combination of hydrophilic and hydrophobic polymers, providing controlled release of metoprolol; and
b) hydrochlorothiazide in an immediate release form.
